(19) United States
(12) Patent Application Publication
(10) Pub. No.: US 2026/0317362 A1
(43) Pub. Date: Oct. 15, 2026

(54) Methods for Increasing Recovery of Allulose from Mixed Allulose/Fructose Solutions

(71) Applicant: CORN PRODUCTS DEVELOPMENT, INC., WESTCHESTER, IL (US)

(72) Inventors: Christopher BELLOVIN, Bridgewater, NJ (US); Shawn YOON, Bridgewater, NJ (US)

(21) Appl. No.: 19/485,600

(22) PCT Filed: May 22, 2024
(86) PCT No.: PCT/US2024/030472

§ 371 Date: Nov. 18, 2025

Related U.S. Application Data
(60) Provisional application No. 63/504,457, filed on May 26, 2023.

(51) Int. Cl. C07H 3/02; C07H 1/06; C12N 9/92

(52) U.S. Cl. CPC C07H 3/02; C07H 1/06; C12N 9/92; C12Y 501/03; C12Y 503/01018

(57) ABSTRACT

The technology disclosed in this specification pertains to methods for making an allulose syrup providing a solution comprising allulose and a borate moiety adding a fructose to the solution to form a mixed material solution separating the allulose from the other components of the mixed material solution to obtain a allulose syrup. The addition of fructose to the form the mixed material is shown in improve allulose recovery. In at least some embodiments the technology further comprises using a glucose isomerase to reduce the amount of glucose in a feed stock solution or recycled raffinate stream.

This specification discloses methods for making allulose. More specifically, this specification discloses methods for improving the recovery of allulose from mixed allulose/fructose solutions.

Allulose is a common name for D-psicose, which is an epimer of fructose. Methods for making allulose from fructose using a D-psicose 3-epimerase are known in the art. The resulting solutions comprise both allulose and fructose. The art also discloses methods for separating allulose from fructose. One method forms allulose-borate complexes and uses simulated moving bed chromatography to recover allulose. A problem arises, however, because the separation of allulose from borate during chromatography is incomplete. This leaves unrecovered allulose in the raffinate stream reducing allulose yield.

### BRIEF DESCRIPTION OF THE FIGURES

The technology disclosed in this specification can be better understood with reference to the following FIGURES, which are not intended to limit the full scope of the technology as disclosed in this specification.

FIG. 1 provides a flow diagram depicting an illustrative an embodiment of the methods described in this specification.

In one aspect the technology disclosed in this specification pertains to methods for improving the recovery of allulose of from a mixed material solution comprising allulose, a boron moiety, and fructose. In embodiments the boron moiety may exist free within solution, complexed with allulose, and complexed with fructose or some combination thereof. In another aspect the method comprises providing a solution comprising allulose and a borate moiety, adding a fructose to the solution to form a mixed material solution, and separating the allulose from the other components of the mixed material solution to obtain a allulose syrup.

In any embodiment of the methods described in this specification, the ratio of allulose to fructose in the mixed material solution is greater than about 1:1.5 (allulose to fructose) or from about 1:1.5 to about 1:4 or from 1:1.5 to about 1:3, or from about 1:1.5 to about 1:2.75, or from about 1:1.5 to about 1:2.5 or from about 1:1.75 to about 1:2:25. With this specification and pertaining to the ratio of allulose to fructose, allulose includes free allulose and allulose borate complexes and fructose includes free fructose and fructose borate complexes

In any embodiment of the method described in this specification, the borate moiety is in an amount from about 1% to about 15% (wt. %) of the solution, or from about 1% to about 10%, or from about 1% to about 9%, to about 8%, or to about 7% or to about 6% or to about 5% or from about 1% to about 5%, or from about 1% to about 4%, or from about 2% to about 4%. Within this specification, pertaining to the borate moiety, the weight percent of borate in the solution includes all borate in whatever for in the solution. This includes the dissolved and undissolved borate, borate complexed or bound with allulose, borate complexed or bound with fructose, and borate associated with, complexed, or bound to another molecule, atom, or ion.

In any embodiment of the method described in this specification, the allulose content of the mixed material solution is from about 5% to about 30% or from about 7.5% to about 30%, or from about 10% to about 30% or from about 12.5% to about 30%, or from about 5% to about 25% or from about 7.5% to about 25%, or from about 10% to about 25% or from about 12.5% to about 25%, or from about 5% to about 20% or from about 7.5% to about 20%, or from about 10% to about 20% or from about 12.5% to about 20% (wt. % of the mixed material). Within this specification, pertaining to allulose content of the mixed material solution, the allulose includes free allulose and allulose borate complexes.

In any embodiment of the method described in this specification, the allulose content of the allulose syrup is from about 90%, or from about 91% or from about 92% or from about 93% or from about 94% or from about 95% or from about 96% or from about 97% to 99.5% (wt. % of the syrup). Within this specification, pertaining to the allulose content of the syrup, allulose content refers all allulose in the syrup, which is substantially free allulose, but may still contain residual allulose borate complexes.

In any embodiment of the method described in this specification, the fructose is added to form the mixed material solution as a fructose syrup having a fructose content relative to its dry mass of is greater than 90%.

In any embodiment of the method described in this specification, the separation step uses a divalent cation resin, optionally wherein, optionally, the cations are selected from the group consisting of calcium, strontium, and barium. In at least some embodiments the divalent cation resin is a calcium resin.

In any embodiment of the method described in this specification, the separation step comprises passing the mixed material solution through a chromatographic system. In any embodiment of the method described in this specification, the separation step involves passing the mixed material solution through a simulated moving bed.

In any embodiment of the method described in this specification the borate compound is selected from the group consisting of boric acid, sodium borate, potassium borate and mixtures thereof. Other borate salts may also be useful such as calcium borate salts and aluminum borate salts.

In any embodiment of the method described in this specification, the separation step obtains an allulose solution and further obtains a fructose and borate solution, and the fructose and borate solution is recycled as a starting material in the method.

In another aspect of the technology a starting material may be glucose instead of fructose. In such embodiment the glucose is converted to fructose using a glucose isomerase and fructose is converted to allulose using a D-psicose-3-epimerase. In various embodiments one or more of the glucose isomerase and D-psicose-3-epimerase is free in solution within the reaction tank. In various other embodiments one or more of the glucose isomerase and D-psicose-3-epimerase are immobilized. Glucose isomerase and D-psicose-3-epimerase are known and commercially available enzymes. The methods described in this specification are not limited as to the source of the glucose isomerase and D-psicose-3-epimerase.

In any embodiment of the method described in this specification, the separated allulose solution may be recovered for use as an allulose syrup. Allulose syrups may be further processed, for example, by further purification steps such as decoloring using activated carbon or other decolorant. Allulose syrups may be concentrated to have a desired solids concentration. Allulose syrups may be blended with other ingredients such as buffers or stabilizers to maintain a desired pH or to prevent crystallization in the syrup.

In any embodiment of the method described in this specification, the allulose syrup is further processed to obtain a solid allulose product, which may be obtained using known crystallization steps or may be obtained by spray drying, freeze drying, or other method of removing water from the syrup to obtain a dry product.

In syrup or solid embodiments the allulose may be blended with other sweeteners or flavor modifiers. Illustrative sweeteners include nutritive sweeteners like sucrose, fructose, and glucose, dextrins and maltodextrins, corn syrups, high fructose corn syrups and blends thereof. Other useful sweeteners include non-nutritive sweeteners like stevia, rebaudiosides, steviolglycosides, erythritol, xylitol, and sucralose.

Allulose syrups, dry allulose sweeteners, and sweetener blends made therefrom can be used as table sweeteners or sweetener syrups by themselves or can be combined with other ingredients to form a sweetened composition.

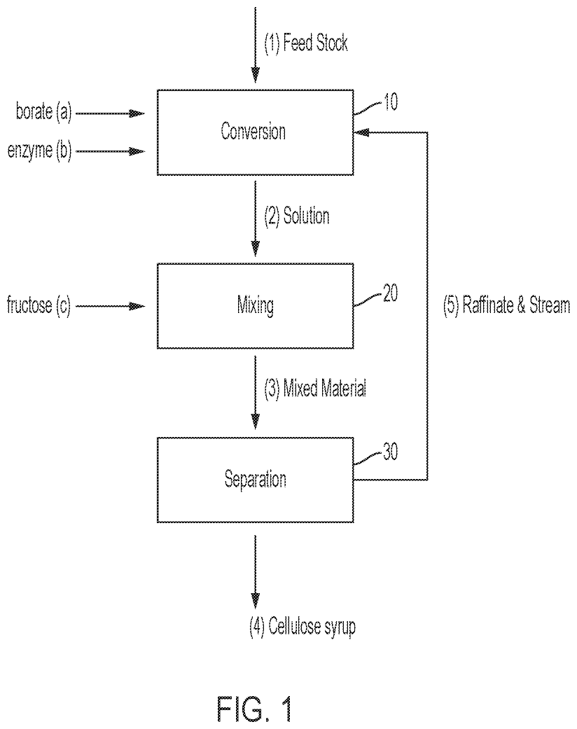

The method described in this specification can be further understood with reference to the flow chart presented in FIG. 1. In step a 10 a feedstock (1) is converted to a solution (2), which comprises allulose and a borate moiety. The feedstock (1) can be a substantially glucose or a fructose feedstock or some combination thereof. The conversion step includes the addition of a borate moiety (a) and one or more enzymes (b). In processes that use only a D-psicose-3-epimerase it is advantageous that the feedstocks (1) have high fructose content, for example having at least 90% by weight or fructose. When using feeds stocks (1) having higher glucose content it is advantageous (although not necessary) to use a glucose isomerase. For example, lower grade high fructose corn syrups, such as 55% fructose syrup (wt. % of the syrup) or 42% fructose syrups (wt. % of the syrup), are more usefully used in the process if glucose isomerase is used to convert glucose in the syrups to fructose so that the fructose can be converted to allulose using a D-psicose-3-epimerase.

In step 10 borate compounds (as described in this specification) form complexes with fructose to improve the conversion rate of fructose to allulose. Borate compounds can be added before, during, or after conversion of feed stock to allulose. In at least some embodiments, borate is added before adding enzymes. Depending on the feedstock the conversion to the solution comprising allulose and a borate moiety is done by contacting the feedstock with a D-psicose-3-epimerase or glucose isomerase or both. If glucose is used as a feedstock the contacting may be sequentially, for example introducing the glucose isomerase first, allowing the reaction to run substantially to completing and adding D-psicose-3-epimerase. Alternately, using a glucose feedstock contacting D-psicose-3-epimerase and glucose isomerase with glucose can be substantially simultaneous. It is noted that use of glucose isomerase does not preclude use of fructose containing syrups.

The conversion of the feedstock (1) to the solution comprising allulose and a borate moiety is done under conditions to optimize efficacy of the variant of the glucose isomerase and D-psicose-3-epimerase from a particular vendor. For example the feedstock may be adjusted to a preferred pH (e.g. to a basic pH from 7.5 to 9 using NaOH or other suitable caustic) or to a desired temperature (e.g. 40° to 50° C.).

In a step 20, once the feedstock (1) has been substantially converted to a solution (2), which comprises allulose and a borate moiety, fructose is added to form a mixed material (3). Fructose is added in so that the mixed material has fructose/allulose ratios in the amounts described in this specification. For simplicity, within this specification, mixed material (3) refers to a collection of materials that exist within the process between step 20 and step 30. Accordingly, the mixed material includes a solution that comprises some combination of borate, allulose, allulose-borate complexes, fructose, and fructose-borate complexes where there is more fructose (and/or fructose-borate complexes) than allulose (and/or allulose-borate complexes).

In a step 30, allulose is separated from the mixed material (3). The separation step 30 comprises one or more separation steps where allulose-borate complexes are broken and the allulose is separated from the other component in the mixed material (3). Borate complexes can be broken, for example using, a divalent ion resin, such as a calcium resin and a chromatography separation system, such as a simulated moving bed chromatography or sequential simulated moving bed (“SSMB”). In some embodiments using SSMB technology fructose can be added to mixed with solution (2) to form mixed material (3) before adding mixed material (3) to the SSMB and the components of mixed material (30). In other embodiments the solution (2) can be added to the SSMB prior to adding fructose, but fructose is directly into the SSMB. In these embodiments mix material (3) forms in the SSMB. As described in this specification, running a mixed material (3) having more fructose than allulose through the separation step improves allulose yield. Without being bound by theory it is believed that the higher concentration of fructose present during chromatographic separation promotes ligand exchange with the allulose-borate, forming fructo-borate and free allulose.

Following separation step 30 two process streams are created, an allulose syrup (4) and a raffinate stream (5). The allulose syrup (4) may be further processed as described in this specification for example to decolor the solution or otherwise prepare it as a commercial product. The allulose syrup, as described in this specification can be blended with other ingredients or can be dried to obtain a desired final product. The raffinate stream (5) can be recycled into the feedstock (1). As content of allulose syrup increases (4), the raffinate stream (5) is comparatively depleted of allulose.

For systems that recycle the raffinate stream (5) into the feedstock stream (1) it is advantageous to use a glucose isomerase to convert any glucose that forms in the raffinate back into fructose. In these systems the feed stock will contain some amount of glucose, fructose, and borate.

It is noted that the solution comprising allulose and a borate moiety (2) can be distinguished from the feedstock (1) because the solution comprising allulose and a borate moiety contains allulose as the predominate sugar because it has been contacted with D-psicose-3-epimerase and exists prior to addition of fructose to make the mixed material (3). It is further noted that the mixed material (3) can be distinguished from the feedstock (1) because feedstock (1) is substantially depleted of allulose.

Use of “about” to modify a number is meant to include the number recited plus or minus 10%. Where legally permissible recitation of a value in a claim means about the value. Use of about in a claim or in the specification is not intended to limit the full scope of covered equivalents.

Recitation of the indefinite article “a” or the definite article “the” is meant to mean one or more unless the context clearly dictates otherwise.

While certain embodiments have been illustrated and described, a person with ordinary skill in the art, after reading the foregoing specification, can effect changes, substitutions of equivalents and other types of alterations to the methods, and of the present technology. Each aspect and embodiment described above can also have included or incorporated therewith such variations or aspects as disclosed regarding any or all the other aspects and embodiments.

The present technology is also not to be limited in terms of the aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing aspects only and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

The technology described in this specification can be better understood with reference to the following aspects, which are not intended to limit the full scope of the technology.

1. A method for producing an allulose syrup comprising: a. providing a solution comprising allulose and a borate moiety b. adding a fructose to the solution to form a mixed material solution c. separating the allulose from the other components of the mixed material solution to obtain a allulose syrup.

2. The method of claim 1 wherein the ratio of allulose to fructose in the mixed material solution is greater than about 1:1.5 (allulose to fructose) or from about 1:1.5 to about 1:4 or from 1:1.5 to about 1:3, or from about 1:1.5 to about 1:2.75, or from about 1:1.5 to about 1:2.5 or from about 1:1.75 to about 1:2:25.

3. The method of claim 1 or 2 wherein the borate moiety in the step a) is in an amount from about 1% to about 15% (wt. %) of the solution, or from about 1% to about 10%, or from about 1% to about 9%, to about 8%, or to about 7% or to about 6% or to about 5% or from about 1% to about 5%, or from about 1% to about 4%, or from about 2% to about 4%.

4. The method of any one of claims 1 to 3 wherein the borate moiety is provided by a borate compound is selected from the group consisting of boric acid, sodium borate, potassium borate and mixtures thereof.

5. The method of any one of claims 1 to 4 wherein the allulose content of the mixed material solution is from about 5% to about 30% or from about 7.5% to about 30%, or from about 10% to about 30% or from about 12.5% to about 30%, or from about 5% to about 25% or from about 7.5% to about 25%, or from about 10% to about 25% or from about 12.5% to about 25%, or from about 5% to about 20% or from about 7.5% to about 20%, or from about 10% to about 20% or from about 12.5% to about 20% (wt. % of the mixed material).

6. The method of claim of any one of claims 1 to 5 wherein the allulose content of the allulose syrup is from about 90%, or from about 91% or from about 92% or from about 93% or from about 94% or from about 95% or from about 96% or from about 97% to 99.5% (wt. % of the syrup).

7. The method of any one of claims 1 to 6 wherein the fructose is added to form the mixed material solution as a fructose syrup having a fructose content relative to its dry mass of is greater than 90%.

8. The method of any one of claims 1 to 7 wherein the separating step c) comprises using a divalent cation, optionally wherein the cations are selected from the group consisting of calcium, strontium, and barium, wherein, optionally the divalent cation is calcium.

9. The method of any one of claims 1 to 8 wherein the separating step c) comprises passing the mixed material solution through a chromatographic separation system, wherein, optionally, the separating step c) comprises passing the mixed material solution through a simulated moving bed.

10. The method of any one of claims 1 to 9 further comprising, in a step d) which precedes the step a): contacting a solution comprising fructose and a borate compound with an allulose-3-epimerase to obtain the solution comprising allulose and a borate moiety, wherein, optionally, the borate compound is in the solution of the step d) an amount from about 1% to about 15% (wt. %) of the solution, or from about 1% to about 10%, or from about 1% to about 9%, to about 8%, or to about 7% or to about 6% or to about 5% or from about 10% to about 5%, or from about 1% to about 4%, or from about 2% to about 4%.

11. The method of any one of claims 1 to 10 wherein the separation step further obtains a fructose and borate solution, and the fructose and borate solution is recycled into the solution comprising fructose and a borate compound of the step d).

12. The method anyone of claims 1 to 11 further comprising in a step d), which precedes the step a): contacting a solution comprising fructose and borate solution with a glucose isomerase.

13. The method of anyone of claims 1 to 12 further comprising obtaining solid allulose from the syrup.

14. The method of anyone of claims 1 to 13 further comprising obtaining a solid allulose from the syrup wherein the solid allulose is obtained using a crystallization process.

15. The method of anyone of claims 1 to 14 further comprising obtaining a solid allulose from the syrup wherein the solid allulose is obtained using spray drying.

17. A method for producing an allulose comprising: a. contacting a solution comprising glucose and a borate moiety with a glucose isomerase and an allulose-3-epimerase, to form a solution comprising allulose and a borate moiety; b. adding fructose to the solution comprising allulose and a borate moiety to form a mixed material solution; c. separating the allulose from the other components of the mixed material solution to obtain a allulose syrup.

18. The method of claim 17 wherein the ratio of allulose to fructose in the mixed material solution is greater than about 1:1.5 (allulose to fructose) or from about 1:1.5 to about 1:4 or from 1:1.5 to about 1:3, or from about 1:1.5 to about 1:2.75, or from about 1:1.5 to about 1:2.5 or from about 1:1.75 to about 1:2:25.

19. The method of claims 17 or 18 wherein the borate moiety in the step a) is in an amount from about 1% to about 15% (wt. %) of the solution, or from about 1% to about 10%, or from about 1% to about 9%, to about 8%, or to about 7% or to about 6% or to about 5% or from about 1% to about 5%, or from about 1% to about 4%, or from about 2% to about 4%.

20. The method of any one of claims 17 to 19 wherein the borate moiety is provided by a wherein the borate compound is selected from the group consisting of boric acid, sodium borate, potassium borate and mixtures thereof.

21. The method anyone of claims 17 to 20 wherein the allulose content of the mixed material solution is from about 5% to about 30% or from about 7.5% to about 30%, or from about 10% to about 30% or from about 12.5% to about 30%, or from about 5% to about 25% or from about 7.5% to about 25%, or from about 10% to about 25% or from about 12.5% to about 25%, or from about 5% to about 20% or from about 7.5% to about 20%, or from about 10% to about 20% or from about 12.5% to about 20% (wt. % of the mixed material).

22. The method of anyone of claims 17 to 21 wherein the allulose content of the allulose syrup is from about 90%, or from about 91% or from about 92% or from about 93% or from about 94% or from about 95% or from about 96% or from about 97% to 99.5% (wt. % of the syrup).

23. The method of anyone of claims 17 to 22 wherein the fructose is added to form the mixed material solution as a fructose syrup having a fructose content relative to its dry mass of is greater than 90%.

24. The method of anyone of claims 17 to 23 wherein the separating step c) comprises using a divalent cation, optionally wherein the cations are selected from the group consisting of calcium, strontium, and barium, wherein, optionally the cation is from calcium.

25. The method of anyone of claims 17 to 24 wherein the separating step c) comprises passing the mixed material solution through a chromatographic separation system, wherein optionally the chromatographic separation system is a simulated moving bed.

27. The e method of any one of claims 17 to 26 wherein the glucose is from a fructose syrup comprising glucose, wherein, optionally, the fructose content of the syrup is no more than about 55% fructose (wt. %).

28. The method of claims 17 to 27 wherein the separation step c) further obtains a fructose and borate solution, and the fructose and borate solution is recycled into the solution comprising glucose and a borate moiety.

29. The method of anyone of claims 17 to 28 further comprising obtaining solid allulose from the syrup.

30. The method of anyone of claims 17 to 29 further comprising obtaining a solid allulose from the syrup wherein the solid allulose is obtained using a crystallization process.

31. The method of anyone of claims 17 to 30 further comprising obtaining a solid allulose from the syrup wherein the solid allulose is obtained using spray drying.

32. A product obtained by a process as described in any foregoing claim.

33. A composition comprising the product of claim 32.

The described in this specification can be better understood with reference to the following examples, which are not intended to limit the full scope of the technology.

### EXAMPLE 1

Allulose separation was evaluated using two allulose/fructose starting solutions, which are described in Table 1. One sample separated a syrup having equal parts allulose and fructose. The other sample separated a syrup having 2-parts fructose to 1-part allulose. 1. Starting mock solutions were created by mixing the following reagents, adjusting the pH to 7 with sodium hydroxide solution, and then adjusting the total volume to 100 mL with deionized water.

TABLE 1
Starting Material Mass Balance

| | 1:1 allulose/Fructose | 1:2 allulose/Fructose |
|---|---|---|
| | Starting Solution (g/g) | Starting Solution (g/g) |
| allulose (g) | 27 | 27 |
| Fructose (g) | 27 | 54 |
| Boric acid (g) | 4.65 | 4.65 |
| 25% NaOH solution (g) | 11.5 | 11.9 |
| Deionized water (g) | 53.05 | 34.85 |
| Mass total per 100 mL | 123.2 (g) | 132.4 (g) |

Separation of allulose from solution was done as follows. 550 mL of DOWEX MONSOSPHERE 99/CA310 resin (saturated in water) was added to a jacketed glass column (“column”) (100 cm length, 2.5 cm inner diameter, 550 mL). Degassed deionized water was used as eluent. The column was heated to 60° C. and system flow rate was set to 12.84 mL/min to allow resin to equilibrate with flow for a few hours. Fourteen mL of starting solution was injected into the column for each run. Sample fractions (6.8 mL) were collected during chromatographic separation using a fractionator. Collected fractions were analyzed for Brix solids and conductivity. Samples were then analyzed for TLC densitometry to determine quantity of free allulose collected for each run. Allulose mass solids are determined via TLC densitometry for each fraction, then all allulose fractions are summed and compared with quantity allulose added into column for percent recovery.

Brix was measured using an ATAGO N-20E Hand Refractometer. Deionized water was used for adjusting Brix baseline reading (0%). Four drops of sample were placed on the refractometer for each recovered fraction and analyzed.

Conductivity was measured using a Mettler Toledo FiveGo (FG3) Conductivity Meter with 3-point calibration. Probe was inserted into each 6.8 mL sample fraction for conductivity reading.

TLC samples were prepared by adjusting sample fraction concentrations to 0.5% solids or lower using deionized water (to determine free allulose recovered) or 0.125M sorbitol solution (to determine total allulose injected from starting solution). Diluted samples and standards were spotted on HPTLC silica gel 60 plates (20×10 cm) and dried prior to elution. The TLC plate was then placed in a glass eluent chamber containing Acetonitrile:Water:Ethyl Acetate (85:15:5) solvent as the mobile phase eluent. TLC plates were irrigated 3 times, with drying cycles in between ascents. Dried plates were then stained with a methanol solution containing 0.3% N-(1-naphthyl) ethylene diamine and 5% sulfuric acid, followed by subsequent heating the plate on a TLC plate heater at 130° C. for 8 min. Plates were cooled to room temperature and scanned using an EPSON Artisan 837 scanner, and the images analyzed for densitometry using Image J (Ver. 1.46r) software. TLC densitometry measurements were done as follows: Percent composition free allulose for each fraction and percent composition total allulose from injected starting solution were both determined by dividing total area of the allulose peak by total area of all components. Percent composition of allulose is then converted to mass by multiplying this value by fraction brix and fraction volume, divided by 100. Percent allulose recovery is then determined by summing up all recovered allulose fractions and dividing by total allulose from injected starting solution.

Sample fractions are reported as bed volumes (BV) of eluent that eluted from the column, and BVs of eluent are tracked following the addition of unseparated sugar solution to the column. The BV is calculated as Eluent volume collected/Resin bed volume. The resin bed volume for the example given in Table 2 is 550 mL.

TABLE 2
Allulose Recovery

| Allulose in fractions | | Allulose in Fractions | |
|---|---|---|---|
| from separation of 1:1 | | from Separation of 1:2 | |
| allulose/fructose solution (g/g) | | allulose/fructose solution (g/g) | |
| Sample | Allulose | Sample | Allulose |
| fraction | recovered | fraction | recovered |
| (BV) | (g) | (BV) | (g) |
| 0.6125-0.650 | 0 | 0.5375-0.55 | 0 |
| 0.650-0.70 | 0 | 0.55-0.60 | 0 |
| 0.70-0.75 | 0 | 0.60-0.65 | 0 |
| 0.75-0.80 | 0 | 0.65-0.70 | 0 |
| 0.80-0.85 | 0 | 0.70-0.75 | 0 |
| 0.85-0.90 | 0 | 0.75-0.80 | 0.03 |
| 0.90-0.95 | 0.07 | 0.80-0.85 | 0.06 |
| 0.95-1.0 | 0.11 | 0.85-0.90 | 0.05 |
| 1.0-1.05 | 0.13 | 0.90-0.95 | 0.12 |
| 1.1-1.15 | 0.17 | 0.95-1.0 | 0.14 |
| 1.15-1.2 | 0.21 | 1.0-1.05 | 0.14 |
| 1.2-1.25 | 0.23 | 1.05-1.1 | 0.11 |
| 1.25-2.0 | 0.96 | 1.1-1.15 | 0.22 |
| | | 1.15-1.4375 | 1.93 |

| | | |
|---|---|---|
| allulose recovered (g) | 2.00 | 2.80 |
| allulose added (g) | 3.79 | 3.78 |
| % Recovery allulose | 52.78% | 74.21% |

As seen total allulose recovered and percent of total allulose recovered increased with fructose in syrups have 2-parts fructose to 1-part allulose. It is believed that the higher concentration of fructose present during chromatographic separation promotes ligand exchange with the allulose-borate, forming fructo-borate and free allulose.

1. A method for producing an allulose syrup comprising:
a. providing a solution comprising allulose and a borate moiety
b. adding a fructose to the solution to form a mixed material solution
c. separating the allulose from the other components of the mixed material solution to obtain an allulose syrup.

2. The method of claim 1 wherein the ratio of allulose to fructose in the mixed material solution is greater than about 1:1.5 (allulose to fructose).

3. The method of claim 1 wherein the borate moiety in the step a) is in an amount from about 1% to about 15% (wt. %) of the solution.

4. The method of claim 1 wherein the borate moiety is provided by a borate compound is selected from the group consisting of boric acid, sodium borate, potassium borate and mixtures thereof.

5. The method of claim 1 wherein the allulose content of the mixed material solution is from about 5% to about 30% (wt. % of the mixed material).

6. The method of claim 1 wherein the allulose content of the allulose syrup is from about 90% (wt. % of the syrup).

7. The method of claim 1 wherein the fructose is added to form the mixed material solution as a fructose syrup having a fructose content relative to its dry mass of is greater than 90%.

8. The method of claim 1 wherein the separating step c) comprises using a divalent cation.

9. The method claim 1 wherein the separating step c) comprises passing the mixed material solution through a chromatographic separation system.

10. The method of claim 1 further comprising, in a step d) which precedes the step a): contacting a solution comprising fructose and a borate compound with an allulose-3-epimerase to obtain the solution comprising allulose and a borate moiety.

11. The method of claim 1 wherein the separation step further obtains a fructose and borate solution, and the fructose and borate solution is recycled into the solution comprising fructose and a borate compound of the step d).

12. The method of claim 1 further comprising in a step d), which precedes the step a): contacting a solution comprising fructose and borate solution with a glucose isomerase.

13. The method of claim 1 further comprising obtaining solid allulose from the syrup.

14. The method of claim 1 further comprising obtaining a solid allulose from the syrup wherein the solid allulose is obtained using a crystallization process.

15. The method of claim 1 further comprising obtaining a solid allulose from the syrup wherein the solid allulose is obtained using spray drying.

16. (canceled)

17. A method for producing an allulose comprising:
a. contacting a solution comprising glucose and a borate moiety with a glucose isomerase and an allulose-3-epimerase, to form a solution comprising allulose and a borate moiety;
b. adding fructose to the solution comprising allulose and a borate moiety to form a mixed material solution;
c. separating the allulose from the other components of the mixed material solution to obtain an allulose syrup.

18. The method of claim 17 wherein the ratio of allulose to fructose in the mixed material solution is greater than about 1:1.5 (allulose to fructose).

19. The method of claim 17 wherein the borate moiety in the step a) is in an amount from about 1% to about 15% (wt. %) of the solution.

20. The method of claim 17 wherein the borate moiety is provided by a wherein the borate compound is selected from the group consisting of boric acid, sodium borate, potassium borate and mixtures thereof.

21-33. (canceled)
